(19) United States
(12) Patent Application Publication
(10) Pub. No.: US 2023/0099126 A1
(43) Pub. Date: Mar. 30, 2023

(54) APPARATUS, METHOD AND COMPUTER READABLE STORAGE MEDIUM FOR CLASSIFYING MENTAL STRESS USING CONVOLUTIONAL NEURAL NETWORK AND LONG-SHORT TERM MEMORY NETWORK

(71) Applicant: Industry-Academic Cooperation Foundation, Chosun University, Gwangju (KR)

(72) Inventors: Youn Tae KIM, Daejeon (KR); Jae Hyo JUNG, Gwangju (KR); Min Gu KANG, Gwangju (KR); Si Ho SHIN, Gwangju (KR)

(21) Appl. No.: 17/678,761

(22) Filed: Feb. 23, 2022

(30) Foreign Application Priority Data
Sep. 28, 2021 (KR) 10-2021-0127984

(51) Int. Cl. A61B 5/00; A61B 5/16; A61B 5/318; G06N 3/04

(52) U.S. Cl. CPC A61B 5/7267; A61B 5/165; A61B 5/318; G06N 3/04

(57) ABSTRACT

An apparatus for classifying mental stress includes a sequence folding layer configured to convert a sequence image of an electrocardiogram signal into an image in an array form; a CNN layer configured to generate a feature map by performing a convolution operation on the image in an array form; a sequence unfolding layer configured to convert the generated feature map into a sequence image; a flatten layer configured to convert the converted sequence image into one-dimensional data; and a long-short term memory network layer configured to extract feature values using a weighted value on the converted one-dimensional data; and a classification module configured to classify stress according to the extracted feature values.

### CROSS-REFERENCE TO RELATED APPLICATION(S)

This application claims benefit of priority to Korean Patent Application No. 10-2021-0127984 filed on Sep. 28, 2021 in the Korean Intellectual Property Office, the disclosure of which is incorporated herein by reference in its entirety.

### BACKGROUND

### 1. Field

Example embodiments of the present disclosure relate to an apparatus, a method and a computer readable storage medium for classifying mental stress using a convolutional neural network and a long-short term memory network.

### 2. Description of Related Art

The mental stress of people these days may be a factor causing or contributing to various diseases such as depression, cancer, and cardiovascular disease, and it may be important to periodically monitor and manage such stress. Stress may be a mental and physical reaction which people might feel when they are in an environment to which people find it difficult to adapt, and when people experience excessive stress, stress may adversely affect people while causing or contributing to chronic diseases such as high blood pressure, heart disease, and cancer, and may further cause death. For this reason, it has been important to observe one's stress in modern society.

Methods for measuring stress using biosignals such as electroencephalography, electromyography, blood oxygen saturation (SpO₂), and photoplethysmography have been suggested. However, these measurement methods may have a large amount of signal noise and a great number of channels, such that it may be complicated to measure a stress signal, and an incorrect peak value may be detected, which may be problematic.

By measuring the electroencephalogram signal using support vector machine (SVM), multilayer perceptron (MLP), and naive bayes (NB), 75% and 85.20% accuracy was obtained. However, in these studies, as the number of pieces of training data was 15, which was insufficient, overfitting may occur, and as the number of channels were seven, measuring a stress signal may be complicated and it may take a great deal of time. In the study in which 85% accuracy was obtained by analyzing the EMG signal by SVM, the amplitudes of the same motion were different due to the fine movement of muscles, and there was excessive noise in the signal, such that it was difficult to extract accurate feature points.

Recently, numerous studies for classifying stress using electrocardiography have been conducted for the simplified method of measuring stress signals. There has been a study result in which 89.21% and 84.4% accuracy was obtained using the support vector machine (SVM) algorithm, but it takes time to measure the electrocardiogram signal and there was a lot of noise, such that it was difficult to extract the feature points. There has been a study result in which 75% to 89% accuracy was obtained by extracting the standard deviation for the R-R interval of the heart rate variability (HRV) signal, but in this study, as it takes five minutes to calculate the standard deviation for the R-R interval of the HRV signal, and the difference in the parameter values is small, it may be difficult to determine the stress state. Also, the electrocardiogram waveform for the frequency domain is not accurate, such that it may be difficult to extract feature points, and it may be difficult to directly identify the effect of noise on the human body.

There has been a study result in which 87.39% and 90.19% accuracy was obtained using convolutional neural networks and convolution recurrent neural networks (CRNNs), but in this study, the stress hierarchical structure is complex, and there is excessive noise, such that it may be difficult to increase stress classification accuracy by detecting the Rpeak value. There has been a study result in which 88.13% accuracy was obtained at Epoch 20 using a long-short term memory network, but, due to the excessive noise of the electrocardiogram signal, it may be difficult to calculate the root mean square of the R-R interval.

Also, there has been the study in which mental stress may be detected by measuring the respiratory signal and the electrocardiogram signal, but it may take a longtime to measure the signals because the respiratory signal and the electrocardiogram signal should be measured separately, and it may be impossible to accurately and objectively determine the physiological signals such as heart disease and stress. The basic method is to measure and determine the electrocardiogram signal and the respiratory signal simultaneously.

Currently, many devices for measuring respiration and electrocardiography have been developed, but most such devices are devised for clinical use and are complicated to use, and expensive equipment should be used, which may be problematic.

There has been a study in which a stress state is diagnosed using parameters of heart rate variability, but, as heart rate variability may be different for each person and noise may occur, it may be difficult to calculate the parameters using an R-R interval of an accurate HRV signal, and the weighted values of the parameters of the heart rate variability may need to be calculated multiple times, which is complicated.

### SUMMARY

An example embodiment of the present disclosure is to provide an apparatus, a method and a computer readable storage medium for classifying mental stress using a convolutional neural network and a long-short term memory network which may, as compared to the case in which stress classification is performed using a general CNN or a long-short term memory network alone, improve the time and performance for classifying stress and may prevent overfitting.

According to an example embodiment of the present disclosure, an apparatus for classifying mental stress includes a sequence folding layer configured to convert a sequence image of an electrocardiogram signal into an image in an array form; a CNN layer configured to generate a feature map by performing a convolution operation on the image in an array form; a sequence unfolding layer configured to convert the generated feature map into a sequence image; a flatten layer configured to convert the converted sequence image into one-dimensional data; and a long-short term memory network layer configured to extract feature values using a weighted value on the converted one-dimensional data; and a classification module configured to classify stress according to the extracted feature values.

The electrocardiogram signal may include an electrocardiogram signal in a time domain and an electrocardiogram signal in a frequency domain with respect to the electrocardiogram signal in the time domain.

The electrocardiogram signal in the frequency domain may be a signal converted from the electrocardiogram signal in the time domain using a spectrogram.

The CNN layer may include a first convolution layer configured to generate a first feature map by performing a convolution operation on the image in an array form; a first max pooling layer configured to reduce a dimension of the first feature map by extracting a maximum value of the generated first feature map; a second convolution layer configured to generate a second feature map by performing a convolution operation on the first feature map having a reduced dimension; and a second max pooling layer configured to reduce a dimension of the second feature map by extracting a maximum value of the generated second feature map.

The apparatus may further include a first normalization layer disposed between the first convolution layer and the first max pooling layer and configured to normalize the first feature map; and a second normalization layer disposed between the second convolution layer and the second max pooling layer and configured to normalize the second feature map.

The classification module may include a fully connected layer configured to output an under-stress state and a without-stress state according to the extracted feature values; a softmax layer configured to calculate probabilities of the output under-stress state and the output without-stress state; and a classification unit configured to classify the electrocardiogram signal into an under-stress state or a without-stress state based on the obtained probabilities.

A method of classifying mental stress includes a first operation of converting a sequence image of an electrocardiogram signal into an image in an array form in a sequence folding layer; a second operation of generating a feature map by performing a convolution operation on the image in an array form in a CNN layer; a third operation of converting the generated feature map into a sequence image in a sequence unfolding layer; a fourth operation of converting the converted sequence image into one-dimensional data in a flatten layer; a fifth operation of extracting feature values using a weighted value on the converted one-dimensional data in a long-short term memory network layer; and a sixth operation of classifying stress according to the extracted feature values.

According to another example embodiment of the present disclosure, a computer readable storage medium in which a program for executing the above-described method on a computer is written may be provided.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a block diagram illustrating an apparatus for classifying mental stress according to an example embodiment of the present disclosure;

FIGS. 2A, 2B, 2C and 2D are diagrams illustrating an under-stress state and a without-stress state in a time domain, and an under-stress state and a without-stress state in a frequency domain converted using a spectrogram according to an example embodiment of the present disclosure;

FIG. 3 is a diagram illustrating a confusion matrix of an apparatus for classifying mental stress according to an example embodiment of the present disclosure;

FIG. 4 is a diagram illustrating an ROC curve of an apparatus for classifying mental stress according to an example embodiment of the present disclosure;

FIG. 5 is a diagram illustrating a PR curve of an apparatus for classifying mental stress according to an example embodiment of the present disclosure; and

FIG. 6 is a flowchart illustrating a method of classifying mental stress according to an example embodiment of the present disclosure.

### DETAILED DESCRIPTION

Hereinafter, embodiments of the present disclosure will be described as below with reference to the attached drawings.

It is to be understood that various equivalents and modifications may replace the example embodiments and configurations at the time of the present application. In the drawings, same elements will be indicated by same reference numerals. Also, redundant descriptions and detailed descriptions of known functions and elements that may unnecessarily make the gist of the present disclosure obscure will be omitted. In the accompanying drawings, some elements may be exaggerated, omitted or briefly illustrated, and the sizes of the elements do not necessarily reflect the actual sizes of these elements.

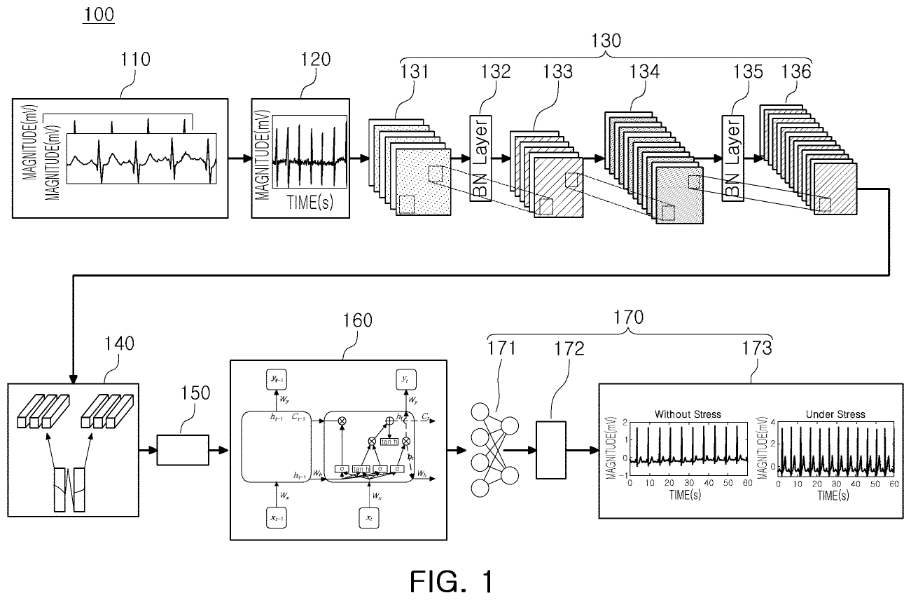

FIG. 1 is a block diagram illustrating an apparatus 100 for classifying mental stress according to an example embodiment.

As illustrated in FIG. 1, the apparatus 100 for classifying mental stress according to an example embodiment may include a sequence input layer 110, a sequence folding layer 120, a CNN layer 130, and a sequence unfolding layer 140, a flatten layer 150, a long-short term memory network layer 160, and a classification module 170.

Specifically, when an electrocardiogram signal in the time domain and the frequency domain is input, the sequence input layer 110 may convert the electrocardiogram signal into a sequence image and may transmit the sequence image to the sequence folding layer 120.

In the example embodiment, the electrocardiogram signal may include an electrocardiogram signal in the time domain and an electrocardiogram signal in the frequency domain with respect to the electrocardiogram signal in the time domain, and the electrocardiogram signal in the frequency domain may be converted from an electrocardiogram signal in the time domain using a spectrogram.

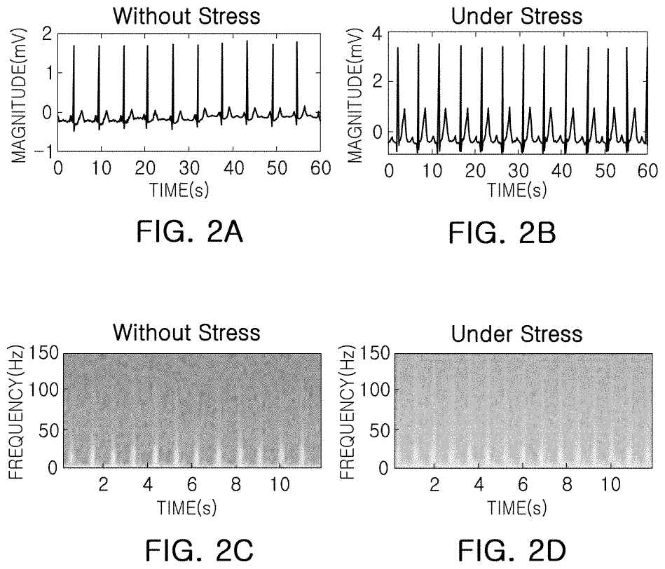

FIGS. 2A, 2B, 2C, 2D are diagrams illustrating an under-stress state and a without-stress state in a time domain, and an under-stress state and a without-stress state in a frequency domain converted using a spectrogram with respect to the under-stress state and a without-stress state in a time domain, respectively, according to an example embodiment.

FIG. 2A illustrates an electrocardiogram signal in the time domain in the without-stress state, FIG. 2C illustrates an electrocardiogram signal in the frequency domain converted from an electrocardiogram signal in the time domain in the without-stress state using a spectrogram, FIG. 2B illustrates an electrocardiogram signal in the time domain in the under-stress state, and FIG. 2D illustrates an electrocardiogram signal in the frequency domain converted from an electrocardiogram signal in the time domain in the under-stress state using a spectrogram.

As illustrated in FIGS. 2A, 2B, 2C, 2D generally, in the state in which the electrocardiogram signal is exposed to noise, the heart may beat irregularly and fast, the R-R Interval of the electrocardiogram signal may become narrow, and [symbol] may decrease. An average [symbol] of the electrocardiogram signal in the resting state was 1.47 mv, and an average [symbol] of the electrocardiogram signal exposed to noise was 4.25 mV.

As illustrated in FIGS. 2A 2B, 2C, 2D, the apparatus for classifying mental stress in the example embodiment may use the electrocardiogram signal in the frequency domain converted using the spectrogram together with the electrocardiogram signal in the time domain for learning, such that the training data set may increase and overfitting may be prevented.

The ST change database and WESAD database were used for learning, and the above-mentioned ST change database may be electrocardiogram data in which physical stress is written, and was data obtained by acquiring 28 electrocardiogram signals from 15 subjects. The WESAD database includes 30 electrocardiogram signals measured from the wrist and chest of 15 subjects.

The sequence folding layer 120 may convert the sequence image of the electrocardiogram signal into an image in an array form. The transformed image in an array form may be transmitted to the CNN layer 130.

The convolutional neural network (CNN) layer 130 may generate a feature map by performing a convolution operation on an image in an array form.

Specifically, the CNN layer 130 may include a first convolution layer 131 configured to generate a first feature map by performing a convolution operation on the image in an array form, a first max pooling layer 133 configured to reduce the dimension of the first feature map by extracting a maximum value of the generated first feature map, a second convolution layer 134 configured to generate a second feature map by performing a convolution operation on the first feature map having the reduced dimension, and a second max pooling layer 136 configured to reduce the dimension of the second feature map by extracting a maximum value of the generated second feature map.

Also, according to an example embodiment, a first normalization layer 132 configured to normalize the first feature map may be further included between the first convolution layer 131 and the first max pooling layer 133, and a second normalization layer 135 configured to normalize the second feature map may be further included between the second convolution layer 134 and the second max pooling layer 136.

The sequence unfolding layer 140 may convert the generated feature map into a sequence image. The converted sequence image may be transferred to the flatten layer 150.

The flatten layer 150 may convert the converted sequence image into one-dimensional data. The converted one-dimensional data may be transferred to the long-short term memory network layer 160

According to an example embodiment, using the one-dimensional data output from the flatten layer as an input value of the long-short term memory network layer 160, it may not be necessary to convert the parameters of the long-short term memory network layer.

The long-short term memory network layer 160 may extract a feature value using a weighted value in the converted one-dimensional data. The extracted feature value may be transmitted to the classification module 170.

By applying the weighted values of Wx=800×11532 and Wh=800×200 to the input layer, a feature value may be extracted by Equation 1 as below:

$$\text{?} \qquad [ Equation 1 ]$$

$${i}_{t} = σ ( \text{?} + \text{?} + \text{?} )$$

$${g}_{t} = tanh ( \text{?} + \text{?} + \text{?} )$$

$${f}_{t} = σ ( \text{?} + \text{?} + \text{?} )$$

$$\text{?} = σ ( \text{?} + \text{?} + \text{?} )$$

$$\text{?} = \text{?}$$

$$\text{?} \text{indicates text missing or illegible when filed}$$

Equation 1 represents the operation process of the long-short term memory network layer 160. The long-short term memory network layer 160 may include an input gate (iₜ, gₜ), an oblivion gate (fₜ), and an output gate (Oₜ). The input gate (iₜ, gₜ) may be configured to determine new information, the oblivion gate (fₜ) may be configured to determine previous information, and the output gate (aₜ) may be configured to control the output value of the updated cell. In this case, each gate may extract the feature value by multiplying the weighted value depending on an input vector (xₜ), a hidden state (hₜ₋₁) and a cell state (Cₜ) using a sigmoid function and a hyperbolic tangent function.

Thereafter, the feature value calculated in the output gate may be transferred to the output layer by applying Equation 2 as below. Equation 4 indicates a process of extracting a necessary feature value by discarding an unnecessary value among the plurality of feature values calculated by the output gate. After the feature values from −1 to 1 is extracted using the Tanh function, the feature values in the corresponding range calculated in the output gate may be transferred to the output layer.

hₜ=oₜ·tanh(cₜ) [Equation 2]

The above-described long-short term memory network layer 160 may be a type of recurrent neural networks (RNNs), and may be an artificial neural network recognizing patterns in data in an array form, such as text, gene signal analysis, and the like. In general artificial neural networks, when data is input, computation may be performed in the input layer and the hidden layer and may be output in sequence. In this process, the input data may pass through the entire nodes only once, and previous data may not be retained.

However, differently from a general artificial neural network, the RNN may be connected such that the result of the hidden layer may go back to the input of the same hidden layer. Therefore, the output of the hidden layer may be repeatedly input to the same hidden layer. However, as large data may not be processed well, a computation speed may be relatively slow. To address this issue, a long-short term memory network (LSTM) may be used. The long-short term memory network may be a special kind of RNN, and may be designed to remember and learn well even when the distance between sequential input data is relatively long.

Finally, the classification module 170 may classify the stress according to the extracted feature value.

Specifically, the classification module 170 may include a fully connected layer 171 configured to outputting an under-stress state and a without-stress state according to the extracted feature value, a softmax layer 172 configured to calculate a probability of each of the output under-stress state and the output without-stress state, and a classification unit 173 configured to classify the electrocardiogram signal into an under-stress state or a without-stress state based on the obtained probability.

The structure of each layer described above is summarized in Table 1 below:

TABLE 1

| Number | Layer | Activations | Weights | Bias |
|---|---|---|---|---|
| 1 | Sequence Input Layer | 124 [symbol] 124 × 3 | — | — |
| 2 | Sequence Folding Layer | 124 [symbol] 124 [symbol] 1 | — | — |
| 3 | Convolution 2D Layer | 124 [symbol] 124 [symbol] 6 | 5 [symbol] 5 [symbol] | 1 [symbol] 1 [symbol] 6 |
| 4 | Batch Normalization | 124 [symbol] 124 [symbol] 6 | — | — |
| | Layer | | | |
| 5 | Max Pooling Layer | [symbol] | — | — |
| 6 | Convolution 2D Layer | 62 [symbol] 12 | [symbol] 12 | 1 [symbol] 1 [symbol] 12 |
| 7 | Batch Normalization | 62 [symbol] 12 | — | — |
| | Layer | | | |
| 8 | Max Pooling Layer | 31 [symbol] 31 [symbol] 12 | — | — |
| 9 | Sequence Unfolding | [symbol] 1 [symbol] 1 [symbol] 12 | — | — |
| | Layer | | | |
| 10 | [symbol] ten Layer | 11 [symbol] | — | — |
| 11 | LSTM Layer | 200 | Input: [symbol] × [symbol] | 800 [symbol] 1 |
| | | | [symbol] | |
| | | | 800 [symbol] 200 | |
| 12 | Fully Connected Layer | 2 | 2 [symbol] 200 | 2 [symbol] 1 |
| 13 | Softmax Layer | 2 | — | — |
| 14 | Classification | — | — | — |

[symbol]

indicates data missing or illegible when filed

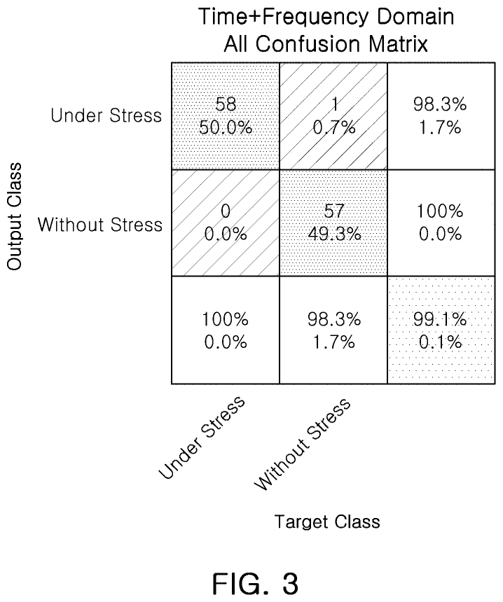

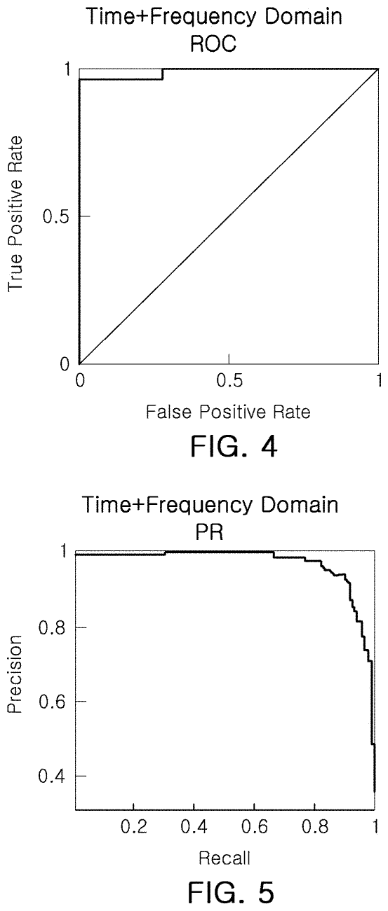

FIG. 3 is a diagram illustrating a confusion matrix of an apparatus for classifying mental stress according to an example embodiment. FIG. 4 is a diagram illustrating an ROC curve of an apparatus for classifying mental stress according to an example embodiment. FIG. 5 is a diagram illustrating a PR curve of an apparatus for classifying mental stress according to an example embodiment.

That is, in the example embodiment, a confusion matrix (see FIG. 3), an ROC curve (see FIG. 4), and a PR curve (see FIG. 5) were used to evaluate the performance of classification of the stress signals.

The confusion matrix may be an index used for evaluating the performance of a model, and may be a matrix indicating how accurately a predicted value predicted an actual observed value.

TABLE 2
Time + Frequency Domain

| | | | | Negative | |
|---|---|---|---|---|---|
| | | | | Predictive | |
| Stress | Precision | Sensitivity | Specificity | Value | Accuracy |
| Performance | 100% | 98.3% | 100% | 98.3% | 99.1% |
| (%) | | | | | |
| Error (%) | 0.0% | 1.7% | 0.0% | 1.7% | 0.9% |

Table 2 lists the values of accuracy, sensitivity, specificity, precision, negative predictive value. Equation 5 below represents a process of calculating accuracy, sensitivity, specificity, precision, and negative predictive value.

$$Accuracy = \frac{TP + TN}{TP + TN + FP + FN} \qquad [ Equation 3 ]$$

$$Sensitivity = \frac{TP}{FN + TP}$$

$$Specificity = \frac{TN}{TN + FP}$$

$$Precision = \frac{TP}{TP + FP}$$

$$Negative Predicitive Value = \frac{TN}{TN + FN}$$

Accuracy indicates the probability of correctly classifying the electrocardiogram signal exposed to all noise and the electrocardiogram signal in a resting state. The accuracy for the time domain and frequency domain was 99.1%. Sensitivity indicates the probability that the algorithm correctly classifies the electrocardiogram signal exposed to noise among the electrocardiogram data exposed to noise as an electrocardiogram signal exposed to noise. The sensitivity in the time domain and the frequency domain was 98.3%. Specificity indicates the probability that the algorithm correctly classifies the electrocardiogram data in the resting state as electrocardiogram data in the resting state. The specificity in the time domain and the frequency domain was 100%. Precision indicates the probability that an algorithm correctly classifies the electrocardiogram data exposed to noise as the electrocardiogram data exposed to noise. The precision in the time domain and the frequency domain was 100%. Negative predictive value indicates the probability that the algorithm correctly classifies the electrocardiogram data exposed to noise when the result is electrocardiogram data exposed to noise. The negative predictive value in the time domain and the frequency domain was 98.3%.

As illustrated in FIG. 5, the stress signal classification accuracy of the algorithm obtained by combining the convolutional neural network with the long-short term memory network according to an example embodiment was 99.1%, and this result is 15.5% of improvement as compared to the algorithm of stress signal classification in the general case in which the CNN is used alone or the long-short term memory network is used alone.

Table 3 below lists a mean squared error depending on the number of epochs in the time domain and the frequency domain to evaluate the performance of classification of the electrocardiogram signal.

TABLE 3
Time + Frequency Domain

| Epoch | MAE |
|---|---|
| 50 | 0.34 |
| 100 | 0.33 |
| 150 | 0.31 |
| 200 | 0.29 |
| 223 | 0.27 |

The performance of stress classification of an algorithm obtained by combining the convolutional neural network with the long-short term memory network with respect to training, validation, and test performance was evaluated. The training data was used to learn the algorithm of the stress signal classification, the validation data was used to determine data values based on the performance of the training data, and the test data was used to evaluate the overall performance of classification of the stress signals. In the time domain and the frequency domain, the error rate of classification of the stress signals was the smallest in Epoch 223 and the performance of classification of the stress signals was excellent.

The receiver operating characteristic (ROC) curve illustrated in FIG. 4 is a performance evaluation technique of a binary classification system, and is an analysis method for determining the presence of a disease such as stress. The area under the curve (AUC), which is the area below the ROC Curve, is an index for evaluating the performance of classification of stress signals. When the AUC range is 0.9 or more and less than 1.0, the performance of classification of the stress signals is excellent, and when the AUC range is 0.8 or more and less than 0.9, the performance of classification of the stress signals is low.

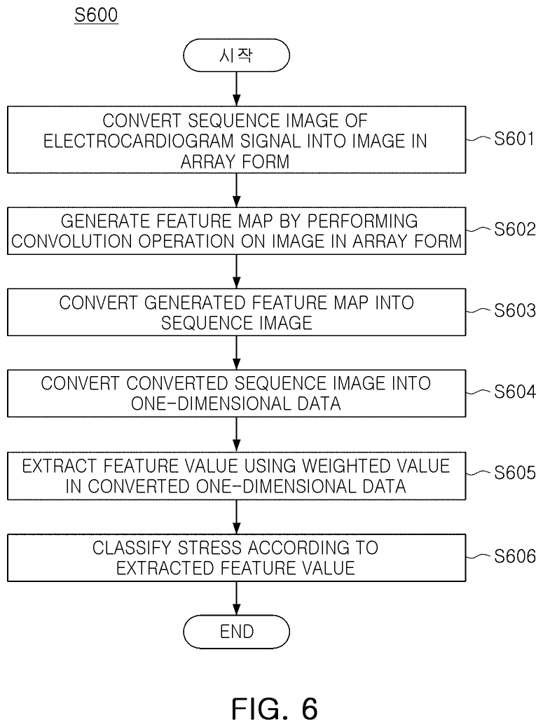

The AUC of the ROC Curve in the time domain and frequency domain in FIG. 6 was 98.3%, whereas, the AUC of the stress signal using the ROC curve was 85.7% in the case in which the CNN is used alone or the long-short term memory network is used alone. Therefore, it is indicated that the AUC of the ROC curve was improved by 12.6% as compared to the general stress signal classification algorithm, and thus that the performance of classification of the stress signals is better.

FIG. 5 illustrates a precession recall (PR) curve according to epoch values in the time domain and the frequency domain using the electrocardiogram signal.

The ROC curve may have difficulty in evaluating the performance of classification of the stress signals because the shape of the curve may be biased to one side when the data set is unstable. The PR curve may be used to overcome the shortcomings of the ROC curve and indicates the relationship between precision and recall. The average precision (AP) of the PR Curve is an index for evaluating the performance of classification of the stress signals. The X axis represents recall (sensitivity), and the Y axis represents precision. In the PR Curve, the larger the AP, the better the performance of classification of the stress signals may be.

In the time domain and frequency domain in FIG. 5, the AUC of the PR curve was 97.6%, and the AP of the general stress signal classification algorithm was 84.2%. Therefore, it is indicated that the AP of the PR curve is improved by 13.4% as compared to the general stress signal classification algorithm, and thus that the stress classification performance is better.

Table 4 below lists the accuracy of classification stress signals using an algorithm obtained by combining a convolutional neural network with a long-short term memory network.

TABLE 4

| | Time + Frequency Domain |
|---|---|
| Epoch | 20 |
| Batch Size | 64 |
| Elapsed Time | 7 min 26 sec |
| Accuracy | 99.1% |

The general stress signal classification algorithm using the time domain and the frequency domain of the electrocardiogram data was determined to Epoch=10, Batch Size=64. As a result, the accuracy in the time domain and the frequency domain for the general stress signal classification algorithm was 83.6% and 74.5%. However, overfitting may occur in this structure in the process of classifying stress signals.

Therefore, in the example embodiment, after determining Epoch=20 and Batch Size=64, accuracy of classification of stress signals for the time domain and the frequency domain of electrocardiogram data was measured using the algorithm obtained by combining a convolutional neural network with a long-short term memory network. As a result of the classification, the classification time in the time domain and the frequency domain was 7 minutes and 28 seconds, and the verification accuracy was 99.1%. Therefore, it is indicated that the accuracy was 15.5% and 24.6% higher than that of the general stress signal classification algorithm.

FIG. 6 is a flowchart illustrating a method of classifying mental stress using a convolutional neural network and a long-short term memory network according to an example embodiment.

Hereinafter, a method for classifying mental stress according to an example embodiment will be described in detail with reference to FIGS. 1 to 6. For ease of description, the description of the portions overlapping those described with reference to FIGS. 1 to 5 will not be provided.

As for the method for classifying mental stress according to an example embodiment, the sequence folding layer 120 may convert a sequence image of an electrocardiogram signal into an image in an array form (S601). The transformed image in an array form may be transmitted to the CNN layer 130.

The convolutional neural network (CNN) layer 130 may generate a feature map by performing a convolution operation on the image in an array form (S602).

Specifically, the CNN layer 130 may include a first convolution layer 131 configured to generate a first feature map by performing a convolution operation on the image in an array form, a first max pooling layer 133 configured to reduce the dimension of the first feature map by extracting a maximum value of the generated first feature map, a second convolution layer 134 configured to generate a second feature map by performing a convolution operation on the first feature map having the reduced dimension, and a second max pooling layer 136 configured to reduce the dimension of the second feature map by extracting a maximum value of the generated second feature map.

Also, according to an example embodiment, a first normalization layer 132 configured to normalize the first feature map may be further included between the first convolution layer 131 and the first max pooling layer 133, and a second normalization layer 135 configured to normalize the second feature map may be further included between the second convolution layer 134 and the second max pooling layer 136.

The sequence unfolding layer 140 may convert the generated feature map into a sequence image (S603). The converted sequence image may be transferred to the flatten layer 150.

The flatten layer 150 may convert the converted sequence image into one-dimensional data (S604). The converted one-dimensional data may be transferred to the long-short term memory network layer 160.

The long-short term memory network layer 160 may extract a feature value using a weighted value in the converted one-dimensional data (S605). The extracted feature value may be transmitted to the classification module 170.

Finally, the classification module 170 may classify stress according to the extracted feature value (S606).

Specifically, as described above, the classification module 170 may include a fully connected layer 171 configured to outputting an under-stress state and a without-stress state according to the extracted feature value, a softmax layer 172 configured to calculate a probability of each of the output under-stress state and the output without-stress state, and a classification unit 173 configured to classify the electrocardiogram signal into an under-stress state or a without-stress state based on the obtained probability.

According to the aforementioned example embodiment, using the output of the CNN layer for generating the feature map as an input to the long-short term memory network layer, and also adding the sequence folding layer, the sequence unfolding layer, and the flatten layer, the classification time and performance of classification of stress may improve as compared to the general case of classification using a CNN or a long-short term memory network alone.

Also, according to an example embodiment, using the electrocardiogram signal in the frequency domain converted using the spectrogram together with the electrocardiogram signal in the time domain during learning, the training data set may increase such that overfitting may be prevented.

Also, the above-described apparatus for classifying mental stress may be used in the development of a variety of medical systems such as home training, sleep state analysis, cardiovascular monitoring, and the like, and may contribute to preventing diseases such as depression, high blood pressure, and diabetes through periodic stress management.

The apparatus and method for classifying mental stress using a convolutional neural network and a long-short term memory network according to an example embodiment described above may be produced as a program to be executed on a computer and may be stored in a computer-readable recording medium. Examples of the computer-readable recording medium may include ROM, RAM, CD-ROM, magnetic tape, floppy disk, optical data storage device, and the like. Also, a computer-readable recording medium may be distributed in a computer system connected through a network, such that the computer-readable code may be stored and executed in a distributed manner. Also, a functional program, a code, and code segments for implementing the method may be easily inferred by programmers in the art to which the present disclosure pertains.

Also, in describing the present disclosure, “ . . . module” and “ . . . unit” may be implemented by various methods, such as, for example, a processor, program instructions executed by a processor, a software module, a microcode, a computer program product, a logic circuit, an application-specific integrated circuit, firmware, or the like.

While the example embodiments have been illustrated and described above, it will be apparent to those skilled in the art that modifications and variations could be made without departing from the scope of the present disclosure as defined by the appended claims.

What is claimed is:

1. An apparatus for classifying mental stress, the apparatus comprising:
a sequence folding layer configured to convert a sequence image of an electrocardiogram signal into an image in an array form;
a CNN layer configured to generate a feature map by performing a convolution operation on the image in an array form;
a sequence unfolding layer configured to convert the generated feature map into a sequence image;
a flatten layer configured to convert the converted sequence image into one-dimensional data; and
a long-short term memory network layer configured to extract feature values using a weighted value on the converted one-dimensional data; and
a classification module configured to classify stress according to the extracted feature values.

2. The apparatus of claim 1, wherein the electrocardiogram signal includes an electrocardiogram signal in a time domain and an electrocardiogram signal in a frequency domain with respect to the electrocardiogram signal in the time domain.

3. The apparatus of claim 1, wherein the electrocardiogram signal in the frequency domain is a signal converted from the electrocardiogram signal in the time domain using a spectrogram.

4. The apparatus of claim 1,
wherein the CNN layer includes:
a first convolution layer configured to generate a first feature map by performing a convolution operation on the image in an array form;
a first max pooling layer configured to reduce a dimension of the first feature map by extracting a maximum value of the generated first feature map;
a second convolution layer configured to generate a second feature map by performing a convolution operation on the first feature map having a reduced dimension; and
a second max pooling layer configured to reduce a dimension of the second feature map by extracting a maximum value of the generated second feature map.

5. The apparatus of claim 4, further comprising:
a first normalization layer disposed between the first convolution layer and the first max pooling layer and configured to normalize the first feature map; and
a second normalization layer disposed between the second convolution layer and the second max pooling layer and configured to normalize the second feature map.

6. The apparatus of claim 1, wherein the classification module includes:
a fully connected layer configured to output an under-stress state and a without-stress state according to the extracted feature values;
a softmax layer configured to calculate probabilities of the output under-stress state and the output without-stress state; and
a classification unit configured to classify the electrocardiogram signal into an under-stress state or a without-stress state based on the obtained probabilities.

7. A method of classifying mental stress, the method comprising:
a first operation of converting a sequence image of an electrocardiogram signal into an image in an array form in a sequence folding layer;
a second operation of generating a feature map by performing a convolution operation on the image in an array form in a CNN layer;
a third operation of converting the generated feature map into a sequence image in a sequence unfolding layer;
a fourth operation of converting the converted sequence image into one-dimensional data in a flatten layer;
a fifth operation of extracting feature values using a weighted value on the converted one-dimensional data in a long-short term memory network layer; and
a sixth operation of classifying stress according to the extracted feature values.

8. A computer readable storage medium in which a program for executing the method in claim 7 on a computer is written.
